(19) United States
(12) Patent Application Publication
(10) Pub. No.: US 2024/0398689 A1
(43) Pub. Date: Dec. 5, 2024

(54) Argan Oil Blend Hair Treatment

(71) Applicant: AIH PERSONAL CARE PRO, LLC, Schaumburg, IL (US)

(72) Inventors: Abdessettar Ibourki, Schaumburg, IL (US); Sharon Martiny, Alton (GB); Khadija Ibourki, Khouribga (MA)

(73) Assignee: AIH PERSONAL CARE PRO, LLC, Schaumburg, IL (US)

(21) Appl. No.: 18/619,748

(22) Filed: Mar. 28, 2024

Related U.S. Application Data
(60) Provisional application No. 63/469,859, filed on May 31, 2023.

(51) Int. Cl. A61K 8/9783; A61Q 5/12

(52) U.S. Cl. CPC A61K 8/9783; A61Q 5/12; A61K 2800/591

(57) ABSTRACT

An all-natural argan oil blend hair treatment is provided that includes a natural oil combination, and an essential oils combination, where the natural oil combination includes argan oil in an amount of about 3% to about 20% by weight of the overall hair treatment formulation. Each of the natural oil combination and the essential oil combination includes multiple all-natural ingredients that work together to moisturize and repair dry, brittle hair and contribute to a shiny, luscious appearance. The argon oil blend hair treatment can be used in combination with an all-natural shampoo and rinse for optimal results.

### RELATED APPLICATIONS

This patent application claims priority to U.S. Provisional Application 63/469,859, filed on May 31, 2023, the disclosure of which is incorporated herein by reference.

### FIELD OF THE INVENTION

This invention is directed to an argan oil blend hair treatment that can be applied as a conditioner prior to or otherwise separate from shampoo. The argan oil blend hair treatment can be applied to hair to help eliminate dry, brittle hair and replace it with healthy hair and luscious locks.

### BACKGROUND OF THE INVENTION

Hair treatment products have been developed over the years with the objective of providing cleaner, shinier, and healthier looking hair. Many of these products contain synthetic ingredients that are effective in shampooing and cleaning the hair, but which can result in dry scalp, dandruff and dry or brittle hair Additionally, the widespread use of synthetic ingredients can result in contamination of rivers, streams, and groundwater, causing harmful effects to fish and other wildlife. There is a need or desire for hair treatment formulas that are not only effective in producing cleaner and shinier hair, but also avoid damage to the users' hair and scalp, and which do not cause harm to the surrounding environment.

### SUMMARY OF THE INVENTION

This invention is directed to an argan oil blend hair treatment formula that can be applied as a hair conditioner prior to shampoo. The argan oil blend hair treatment conditioner contains all natural ingredients that promote healthy, shiny hair with luscious locks, and have environmental advantages.

The argan oil blend hair treatment can include some or all the following ingredients, which can be present in the amounts described herein.

Natural Oils: sunflower oil, canola (rapeseed) oil, argan oil, sesame seed oil, black seed oil, castor oil, coconut oil, vitamin E.

Essential Oils: thyme essential oil, basil leaf essential oil, green tea essential oil, lavender essential oil, linseed essential oil, carrot seed essential oil, Moroccan peppermint essential oil, rosemary essential oil, juniper essential oil, sage dalmatian essential oil, ginger essential oil, pennyroyal essential oil, pomegranate peel essential oil, chamomile maroc essential oil, clove bud essential oil, turmeric essential oil, Moroccan rose absolute essential oil, onion bub essential oil, garlic essential oil.

In one embodiment, the argan oil blend hair treatment includes a natural oil combination and an essential oil combination. The natural oil combination can include argan oil in an amount of about 3% to about 20% by weight, based on the total weight of the argan oil blend hair treatment. The natural oil combination can also include one or more of sunflower oil, canola oil, coconut oil, castor oil, blackseed oil, and natural vitamin E. In this embodiment the argan oil blend hair treatment can include at least about 75% by weight, or at least about 80% by weight, or at least about 85% by weight, or at least about 90% by weight of a natural oil combination and up to about 99% by weight, or up to about 98% by weight, or up to about 97% by weight, or up to about 96% by weight of the natural oil combination. In this embodiment, the argan oil blend hair treatment can include a least about 1% by weight, or at least about 2% by weight, or at least about 3% by weight, or at least about 4% by weight of the essential oil combination, and up to about 25% by weight, or up to about 20% by weight, or up to about 15% by weight, or up to about 10% by weight of the essential oil combination.

In one embodiment, the natural oil combination can include about 20% to about 45% by weight sunflower oil, about 20% to about 45% by weight canola oil, about 3% to about 20% by weight coconut oil, about 3% to about 20% by weight castor oil, and about 3% to about 20% by weight argan oil based on the total weight of the argan oil blend hair treatment. The natural oil combination can also include up to about 5% by weight black seed oil and/or up to about 5% by weight natural vitamin E based on the weight of the argan oil blend hair treatment.

In one embodiment, the essential oil combination can include about 0.05% to about 2% by weight of each of the essential oils that are present in the essential oil combination, based on the total weight of the argan oil blend hair treatment, provided that the overall weight of the essential oil combination is within the ranges stated above.

With the foregoing in mind, it is a feature and advantage of the invention to provide an argan oil blend hair treatment that includes:

about 75% to about 99% by weight of a natural oil combination; and

about 1% to about 25% by weight of an essential oil combination; and

wherein the natural oil combination includes about 3% to about 20% argan oil based on a weight of the argan oil blend hair treatment.

It is also a feature and advantage of the invention to provide an argan oil blend hair treatment that includes:

about 80% to about 98% by weight of a natural oil combination;

about 2% to about 20% by weight of an essential oil combination;

wherein the natural oil combination includes argan oil and one or more of sunflower oil, canola oil, coconut oil, castor oil, black seed oil, and vitamin E.

It is also a feature and advantage of the invention to provide an argan oil blend hair treatment that includes:

about 85% to about 97% by weight of a natural oil combination;

about 3% to about 15% by weight of an essential oil combination;

wherein the natural oil combination includes at least argan oil, sunflower oil, canola oil, coconut oil, and castor oil;

and the essential oil combination includes at least twelve of thyme essential oil, basil leaf essential oil, green tea essential oil, lavender essential oil, linseed essential oil, carrot seed essential oil, Moroccan peppermint essential oil, rosemary essential oil, juniper essential oil, sage dalmatian essential oil, ginger essential oil, pennyroyal essential oil, pomegranate peel essential oil, chamomile maroc essential oil, clove bud essential oil, turmeric essential oil, Moroccan rose absolute essential oil, onion bub essential oil, and garlic essential oil.

Specific embodiments of the argan oil blend hair treatment are described in the following Detailed Description.

### DETAILED DESCRIPTION OF THE INVENTION

The argan oil blend hair treatment is especially designed to provide the user with shiner, healthier hair with a luscious appearance, and is environmentally friendly. The argan oil hair treatment can be used alone as a conditioner, or as part of an overall hair treatment. One example of an overall hair treatment can include shampooing the hair with a herbal cleansing shampoo as described in co-pending U.S. Patent Application entitled “Herbal Cleansing Shampoo” and rinsing the shampoo with a rinse-free nutrient rich rinse as described in co-pending U.S. Patent Application entitled “Rinse-Free Nutrient Rich Rinse.” Both co-pending applications are filed on even date herewith and are incorporated herein by reference.

The argan oil blend hair treatment can include a natural oil combination and an essential oil combination. The natural oil combination can include about 3% to about 20% by weight argan oil, based on the total weight of the argan oil blend hair treatment. The natural oil combination can also include one or more of sunflower oil, canola oil, coconut oil, castor oil, black seed oil, and natural vitamin E. In some embodiments, the natural oil combination can include two or more, three or more, four or more, five or more, or all of sunflower oil, canola oil, coconut oil, castor oil, black seed oil, and natural vitamin E, in addition to the argan oil. For example, the natural oil combination can include argan oil, sunflower oil, and one or more of canola oil, coconut oil, castor oil, black seed oil, and natural vitamin E. The natural oil combination can include argan oil, canola oil, and one or more of sunflower oil, coconut oil, castor oil, black seed oil, and natural vitamin E. The natural oil combination can include argan oil, canola oil, sunflower oil, and one or more of coconut oil, castor oil, black seed oil, and natural vitamin E.

In one embodiment, the natural oil combination can include about 4% to about 15%, or about 5% to about 10% by weight of the argan oil, based on the total weight of the argan oil blend hair treatment. In one embodiment, in addition to the argan oil, the natural oil combination can include about 20% to about 45% sunflower oil, or about 25% to about 40% by weight sunflower oil, or about 30% to about 35% by weight sunflower oil, based on the total weight of the argan oil blend hair treatment. In one embodiment, in addition to the argan oil, the natural oil combination can include about 20% to about 45% canola oil, or about 25% to about 40% by weight canola oil, or about 30% to about 35% by weight canola oil, based on the total weight of the argan oil blend hair treatment. In one embodiment, in addition to the argan oil, the natural oil combination can include about 3% to about 20% coconut oil, or about 5% to about 15% by weight coconut oil, or about 7% to about 12% by weight coconut oil, based on the total weight of the argan oil blend hair treatment. The natural oil combination can include about 3% to about 20% castor oil, or about 5% to about 15% by weight castor oil, or about 7% to about 12% by weight castor oil, based on the total weight of the argan oil blend hair treatment. As indicated above, the natural oil combination can include any combination of, or all of the foregoing additional ingredients. The natural oil combination can also include the foregoing black seed oil and natural vitamin E, each in amounts of about 0.3% to about 4% by weight, or about 0.5% to about 3% by weight, or about 0.7% to about 2% by weight of the argan oil blend hair treatment, and can optionally include additional natural oils in suitable minor amounts.

The essential oil combination can include some or all of the following ingredients: thyme essential oil, basil leaf essential oil, green tea essential oil, lavender essential oil, linseed essential oil, carrot seed essential oil, Moroccan peppermint essential oil, rosemary essential oil, juniper essential oil, sage dalmatian essential oil, ginger essential oil, pennyroyal essential oil, pomegranate peel essential oil, chamomile maroc essential oil, clove bud essential oil, turmeric essential oil, Moroccan rose absolute essential oil, onion bub essential oil, and garlic essential oil. For example, the essential oil combination can include at least four, or at least six, or at least eight, or at least ten, or at least twelve, or at least fourteen, or at least sixteen, or at least eighteen of these ingredients. Each of the essential oils that are included in the essential oil combination can be present in amounts of about 0.05% to about 2.0% by weight, or about 0.08% to about 1.0% by weight, or about 0.10% to about 0.70% by weight, based on the total weight of the argan oil blend hair treatment.

As explained above, the argan oil blend hair treatment can include at least about 75% by weight, or at least about 80% by weight, or at least about 85% by weight, or at least about 90% by weight of the natural oil combination and up to about 99% by weight, or up to about 98% by weight, or up to about 97% by weight, or up to about 96% by weight of the natural oil combination. For example, the argan oil blend hair treatment can include about 75% to about 99% by weight, or about 80% to about 98% by weight, or about 85% to about 97% by weight, or about 90% to about 96% by weight of the natural oil combination.

As explained above, the argan oil blend hair treatment can include a least about 1% by weight, or at least about 2% by weight, or at least about 3% by weight, or at least about 4% by weight of the essential oil combination, and up to about 25% by weight, or up to about 20% by weight, or up to about 15% by weight, or up to about 10% by weight of the essential oil combination. For example, the argan oil blend hair treatment can include about 1% to about 25% by weight, or about 2% to about 20% by weight, or about 3% to about 15% by weight, or about 4% to about 10% by weight of the essential oil combination.

The following Table 1 shows an example of an argan oil blend hair treatment formulation useful in the invention. The ingredients can be added and mixed in the order listed, or in a different order, to produce the argan oil blend hair treatment.

TABLE 1
Example Argan Oil Blend Hair Treatment Formula (% By Weight)

| | Broad | Intermediate | Narrow |
|---|---|---|---|
| Ingredient | Range | Range | Range |
| Natural Oil Combination | 80-98 | 85-97 | 90-96 |
| Sunflower Oil | 20-45 | 25-40 | 30-35 |
| Canola (Rapeseed) Oil | 20-45 | 25-40 | 30-35 |
| Coconut Oil Fractions | 3-20 | 5-15 | 7-12 |
| Castor Oil | 3-20 | 5-15 | 7-12 |
| Argan Oil | 3-20 | 4-15 | 5-10 |
| Black Seed Oil | 0.3-4.0 | 0.5-3.0 | 0.7-2.0 |
| Vitamin E (Natural) | 0.3-4.0 | 0.5-3.0 | 0.7-2.0 |
| Essential Oil Combination | 2-20 | 3-15 | 4-10 |
| Lavender Essential Oil | 0.05-2.0 | 0.08-1.0 | 0.10-0.70 |
| Linseed Essential Oil | 0.05-2.0 | 0.08-1.0 | 0.10-0.70 |
| Carrot Seed Essential Oil | 0.05-2.0 | 0.08-1.0 | 0.10-0.70 |
| Basil Leaf Essential Oil | 0.05-2.0 | 0.08-1.0 | 0.10-0.70 |
| Thyme Essential Oil | 0.05-2.0 | 0.08-1.0 | 0.10-0.70 |
| Moroccan Peppermint Essential Oil | 0.05-2.0 | 0.08-1.0 | 0.10-0.70 |
| Rosemary Essential Oil | 0.05-2.0 | 0.08-1.0 | 0.10-0.70 |
| Juniper Essential Oil | 0.05-2.0 | 0.08-1.0 | 0.10-0.70 |
| Sage Dalmatian Essential Oil | 0.05-2.0 | 0.08-1.0 | 0.10-0.70 |
| Ginger Essential Oil | 0.05-2.0 | 0.08-1.0 | 0.10-0.70 |
| Pennyroyal Essential Oil | 0.05-2.0 | 0.08-1.0 | 0.10-0.70 |
| Pomegranate Essential Oil | 0.05-2.0 | 0.08-1.0 | 0.10-0.70 |
| Chamomile Maroc Essential Oil | 0.05-2.0 | 0.08-1.0 | 0.10-0.70 |
| Clove Bud Essential Oil | 0.05-2.0 | 0.08-1.0 | 0.10-0.70 |
| Turmeric Essential Oil | 0.05-2.0 | 0.08-1.0 | 0.10-0.70 |
| Moroccan Rose Absolute Essential Oil | 0.05-2.0 | 0.08-1.0 | 0.10-0.70 |
| Onion Bulb Essential Oil | 0.05-2.0 | 0.08-1.0 | 0.10-0.70 |
| (10% in Sunflower Oil) | | | |
| Garlic Essential Oil | 0.05-2.0 | 0.08-1.0 | 0.10-0.70 |
| (10% in Sunflower Oil) | | | |
| TOTAL | 100 | 100 | 100 |

Several ingredients of the argan oil blend hair treatment have advantageous properties that, together and alone, contribute to shiny, healthy, thicker, and younger-looking hair and a healthy scalp. Argan oil is rich in fatty acids and antioxidants that help to nourish and protect the hair from damage, leaving it shiny and healthy-looking. It also helps to moisturize and hydrate the hair, making it an excellent choice for dry and damaged hair. Sunflower oil provides the scalp and hair strands with nutrients needed to promote shiny, strong hair. Its hydrating influence promotes scalp and hair health and helps make the hair thicker. Canola oil promotes hair growth and greatly benefits the hair and scalp, thanks to its high fatty acid content of about 60% oleic acid and about 20% linoleic acid. Linoleic acid has a light texture and moisturizes the scalp as well as promoting hair growth.

Coconut oil is rich in fatty acids that help to nourish and moisturize the hair, reducing dryness and breakage. It also has antimicrobial properties that help to prevent scalp infections and dandruff, promoting a healthy and clean scalp. Castor oil is rich in ricinoleic acid, which helps to nourish and strengthen hair follicles, reducing hair fall and promoting healthy hair growth. It also has natural antimicrobial properties that help to prevent scalp infections and dandruff, promoting a healthy scalp. Black seed oil is rich in antioxidants and essential fatty acids that help to nourish and strengthen hair follicles, reducing hair fall and promoting healthy hair growth. It also has anti-inflammatory properties that help to soothe and calm the scalp, reducing irritation and dandruff. Natural Vitamin E is a powerful antioxidant that helps to protect the hair from damage caused by free radicals, promoting healthy hair growth and shine. It also has moisturizing properties that help to nourish and strengthen the hair, reducing dryness and breakage.

Lavender essential oil has antimicrobial properties that help to prevent scalp infections and dandruff, promoting a healthy scalp. It also has a calming effect on the scalp, reducing stress and tension, which can lead to healthier hair growth. Linseed essential oil provides hair with a natural shine and helps seal in moisture. Carrot seed essential oil is a natural source of beta-carotene and vitamin A, which are essential nutrients for healthy hair growth and scalp health. It also contains antioxidants that help protect the hair from damage caused by free radicals, promoting stronger and more lustrous hair. Basil leaf essential oil contains antioxidants that help to nourish and strengthen hair follicles, promoting healthy hair growth. It also has antibacterial properties that help to prevent scalp infections and dandruff, leaving the hair clean and healthy. Thyme essential oil is a natural astringent that helps to cleanse the scalp and prevent buildup of oil and dirt, promoting a healthy scalp. It also contains vitamins and minerals that help to nourish and strengthen hair, reducing hair loss and promoting healthy growth.

Peppermint essential oil has a cooling and refreshing effect on the scalp, reducing itchiness and irritation. It also has astringent properties that help to cleanse the scalp and prevent buildup of oil and dirt, promoting a healthy scalp and reducing dandruff. Rosemary essential oil has stimulating properties that help to promote blood circulation to the scalp, providing nourishment to hair follicles and promoting healthy hair growth. It also has anti-inflammatory properties that help to soothe and calm the scalp, reducing irritation and dandruff. Juniper essential oil possesses antibacterial and antifungal properties that can combat dandruff and itchy scalp. It also helps balance the scalp's oil production. Juniper essential oil possesses antibacterial and antifungal properties that can help combat dandruff and itchy scalp. Sage dalmatian essential oil can help regenerate new hair follicles and can help improve blood circulation to the scalp.

Ginger essential oil has stimulating properties that help to promote blood flow to the scalp, providing nourishment to hair follicles and promoting healthy hair growth. It also has anti-inflammatory properties that help to soothe and calm the scalp, reducing irritation and dandruff. Pennyroyal essential oil can promote hair growth. Pomegranate essential oil is rich in antioxidants that help to protect the hair from damage caused by free radicals, promoting healthy hair growth and shine. It also has anti-inflammatory properties that help to soothe and calm the scalp, reducing irritation and dandruff. Chamomile essential oil has anti-inflammatory properties that soothe and calm the scalp, reducing irritation and dandruff. It also contains antioxidants that help to protect the hair from environmental stressors, promoting healthy hair growth and shine.

Clove bud essential oil contains eugenol, which is a natural antiseptic that helps to prevent scalp infections and dandruff. It also has stimulating properties that help to promote blood flow to the scalp, providing nourishment to hair follicles and promoting healthy hair growth. Turmeric essential oil has anti-inflammatory and antiseptic properties that help to soothe and heal the scalp, reducing irritation, and promoting a healthy scalp. It also contains antioxidants that help to protect the hair from damage caused by free radicals, promoting healthy hair growth and shine. Rose essential oil has natural antiseptic and antimicrobial properties that help to prevent scalp infections and dandruff, promoting a healthy and clean scalp. It also has moisturizing properties that help to nourish and strengthen the hair, reducing dryness and breakage.

Onion bulb essential oil is rich in sulfur and potassium and helps to strengthen hair strands and prevent splitting and breakage. Garlic essential oil is rich in sulfur and selenium, which are essential minerals for healthy hair growth and scalp health. It also has antimicrobial properties that help to prevent scalp infections and dandruff, promoting a healthy and clean scalp.

No special order of addition or mixing is required for blending the ingredients of the argan oil blend hair treatment together. The ingredients can be added and mixed all at once to any suitable agitated mixture, or they can be premixed as separate sub-components that can then be blended together. The ingredients can be mixed thoroughly at ambient temperature and pressure and can be filled into bottles or other suitable containers for use by consumers. In one embodiment, the inion bulb essential oil, garlic essential oil, and sunflower oil can be premixed. The remaining ingredients can be added and thoroughly mixed in the order listed in Table 1.

The argan oil blend hair treatment can be applied to the user's hair, hair roots and scalp approximately every two weeks, or more or less often. Depending on the length of the hair, the argan oil blend hair treatment can be applied in amounts of about one to about three tablespoons or more and can be gently massaged into the hair roots and scalp. The argan oil blend hair treatment can remain in the user's hair for about 1-2 hours to allow optimum absorption into the hair follicles. The user's hair can then be separately shampooed rinsed, for example by using the herbal cleansing shampoo as described in the above-identified co-pending U.S. patent application Ser. No. ______, entitled “Herbal Cleansing Shampoo” and separately rinsing the dry or damp hair with a rinse-free nutrient rich rinse as described in co-pending U.S. patent application Ser. No. ______, entitled “Rinse-Free Nutrient Rich Rinse.”

The embodiments of the invention described herein are exemplary. Various modifications and improvements can be made without departing from the spirit and scope of the invention. The scope of the invention is defined in the appended claims, and all changes that fall within the meaning and range of equivalency are intended to be embraced therein.

1. An argan oil blend hair treatment, comprising:
about 75% to about 99% by weight of a natural oil combination; and
about 1% to about 25% by weight of an essential oil combination; and
wherein the natural oil combination includes about 3% to about 20% argan oil based on a weight of the argan oil blend hair treatment.

2. The argan oil blend hair treatment of claim 1, wherein the natural oil combination further comprises about 20% to about 45% by weight sunflower oil by weight of the argan oil blend hair treatment.

3. The argan oil blend hair treatment of claim 2, wherein the natural oil combination further comprises about 20% to about 45% by weight canola oil by weight of the argan oil blend hair treatment.

4. The argan oil blend hair treatment of claim 3, wherein the natural oil combination further comprises about 3% to about 20% by weight castor oil by weight of the argan oil blend hair treatment.

5. The argan oil blend hair treatment of claim 1, wherein the essential oil combination comprises at least four of thyme essential oil, basil leaf essential oil, green tea essential oil, lavender essential oil, linseed essential oil, carrot seed essential oil, Moroccan peppermint essential oil, rosemary essential oil, juniper essential oil, sage dalmatian essential oil, ginger essential oil, pennyroyal essential oil, pomegranate peel essential oil, chamomile maroc essential oil, clove bud essential oil, turmeric essential oil, Moroccan rose absolute essential oil, onion bub essential oil, and garlic essential oil.

6. The argan oil blend hair treatment of claim 1, wherein the essential oil combination comprises at least eight of thyme essential oil, basil leaf essential oil, green tea essential oil, essential oil, rosemary essential oil, juniper essential oil, sage dalmatian essential oil, ginger essential oil, pennyroyal essential oil, pomegranate peel essential oil, chamomile maroc essential oil, clove bud essential oil, turmeric essential oil, Moroccan rose absolute essential oil, onion bub essential oil, and garlic essential oil.

7. The argan oil blend hair treatment of claim 1, wherein the essential oil combination comprises at least twelve of thyme essential oil, basil leaf essential oil, green tea essential oil, lavender essential oil, linseed essential oil, carrot seed essential oil, Moroccan peppermint essential oil, rosemary essential oil, juniper essential oil, sage dalmatian essential oil, ginger essential oil, pennyroyal essential oil, pomegranate peel essential oil, chamomile maroc essential oil, clove bud essential oil, turmeric essential oil, Moroccan rose absolute essential oil, onion bub essential oil, and garlic essential oil.

8. The argan oil blend hair treatment of claim 1, comprising:
about 80% to about 98% by weight of the natural oil combination; and
about 2% to about 20% by weight of the essential oil combination.

9. The argan oil blend hair treatment of claim 1, comprising:
about 85% to about 97% by weight of the natural oil combination; and
about 3% to about 15% by weight of the essential oil combination.

10. The argan oil blend hair treatment of claim 1, comprising:
about 90% to about 96% by weight of the natural oil combination; and
about 4% to about 10% by weight of the essential oil combination.

11. An argan oil blend hair treatment, comprising:
about 80% to about 98% by weight of a natural oil combination;
about 2% to about 20% by weight of an essential oil combination;
wherein the natural oil combination includes argan oil and one or more of sunflower oil, canola oil, coconut oil, castor oil, black seed oil, and vitamin E.

12. The argan oil blend hair treatment of claim 11, wherein the natural oil combination comprises argan oil, sunflower oil, and one or more of canola oil, coconut oil, castor oil, black seed oil, and vitamin E.

13. The argan oil blend hair treatment of claim 11, wherein the natural oil combination comprises argan oil, canola oil, and one or more of sunflower oil, coconut oil, castor oil, black seed oil, and vitamin E.

14. The argan oil blend hair treatment of claim 11, wherein the natural oil combination comprises argan oil, sunflower oil, canola oil, and one or more of coconut oil, castor oil, black seed oil, and vitamin E.

15. The argan oil blend hair treatment of claim 11, wherein the natural oil combination comprises argan oil, sunflower oil, canola oil, and two or more or more of coconut oil, castor oil, black seed oil, and vitamin E.

16. The argan oil blend hair treatment of claim 11, wherein the natural oil combination comprises argan oil, sunflower oil, canola oil, and three or more or more of coconut oil, castor oil, black seed oil, and vitamin E.

17. The argan oil blend hair treatment of claim 11, wherein the natural oil combination comprises argan oil, sunflower oil, canola oil, coconut oil, castor oil, black seed oil, and vitamin E.

18. The argan oil blend hair treatment of claim 11, wherein the essential oil combination comprises at least six of thyme essential oil, basil leaf essential oil, green tea essential oil, essential oil, rosemary essential oil, juniper essential oil, sage dalmatian essential oil, ginger essential oil, pennyroyal essential oil, pomegranate peel essential oil, chamomile maroc essential oil, clove bud essential oil, turmeric essential oil, Moroccan rose absolute essential oil, onion bub essential oil, and garlic essential oil.

19. The argan oil blend hair treatment of claim 11, wherein the essential oil combination comprises at least twelve of thyme essential oil, basil leaf essential oil, green tea essential oil, lavender essential oil, linseed essential oil, carrot seed essential oil, Moroccan peppermint essential oil, rosemary essential oil, juniper essential oil, sage dalmatian essential oil, ginger essential oil, pennyroyal essential oil, pomegranate peel essential oil, chamomile maroc essential oil, clove bud essential oil, turmeric essential oil, Moroccan rose absolute essential oil, onion bub essential oil, and garlic essential oil.

20. An argan oil blend hair treatment, comprising:
about 85% to about 97% by weight of a natural oil combination;
about 3% to about 15% by weight of an essential oil combination;
wherein the natural oil combination includes at least argan oil, sunflower oil, canola oil, coconut oil, and castor oil;
and the essential oil combination includes at least twelve of thyme essential oil, basil leaf essential oil, green tea essential oil, lavender essential oil, linseed essential oil, carrot seed essential oil, Moroccan peppermint essential oil, rosemary essential oil, juniper essential oil, sage dalmatian essential oil, ginger essential oil, pennyroyal essential oil, pomegranate peel essential oil, chamomile maroc essential oil, clove bud essential oil, turmeric essential oil, Moroccan rose absolute essential oil, onion bub essential oil, and garlic essential oil.
